(19) United States
(12) Patent Application Publication
(10) Pub. No.: US 2016/0022297 A1
(43) Pub. Date: Jan. 28, 2016

(54) METHOD AND APPARATUS FOR LITHIASIS TREATMENT

(71) Applicants: XuLiang Qian, Rancho Cucamonga, CA (US); Kun Qian, Rancho Cucamonga, CA (US)

(72) Inventors: XuLiang Qian, Rancho Cucamonga, CA (US); Kun Qian, Rancho Cucamonga, CA (US)

(21) Appl. No.: 14/807,863

(22) Filed: Jul. 23, 2015

Related U.S. Application Data
(60) Provisional application No. 62/028,084, filed on Jul. 23, 2014.

(51) Int. Cl. A61B 17/22

(52) U.S. Cl. CPC A61B 17/22032; A61B 2017/22035; A61B 2017/22069; A61B 2017/22067; A61B 2017/22054; A61B 2017/22084

(57) ABSTRACT

A medical apparatus may include an inflatable tubular component that can be inflated into a U-shaped structure, a plurality of connecting portions formed on a main body. In one embodiment, the connecting portion can be used for inflating an inflatable cylindrical component formed in the U-shaped structure. The medical apparatus treatment may further include a plurality of control filaments disposed on an inner surface of the U-shaped structure to control the size thereof, so the inner surface of the U-shaped structure can tightly press against an outer surface of the inflatable cylindrical component when it is inflated, and a chamber is formed in the U-shaped structure next to the inflated cylindrical component. When in use, the stones can be trapped into the chamber and chemical reagents can be introduced to either dissolve or disrupt the stones.

### CROSS-REFERENCE TO RELATED APPLICATIONS

This application claims priority under 35 U.S.C. §119 (e) to U.S. Provisional Patent Application Ser. No. 62/028,084 filed on Jul. 23, 2014, the entire contents of which are hereby incorporated by reference.

### FIELD OF THE INVENTION

The present invention relates generally to a medical apparatus and method, and more particularly to a method and apparatus for capturing and dissolving urinary stones from the bladder.

### BACKGROUND OF THE INVENTION

Lithiasis is a common human ailment characterized by calculi or “stones” formed within a human body. Calculi or stones usually arise because of the breakdown of a delicate balance in the body. Bladder stones are small calculi that can form in the bladder. In most cases, stones usually develop when the urine becomes very concentrated or when one is dehydrated. This allows for the minerals like calcium or magnesium to crystallize and form stones.

Although small stones are easily voided from the bladder with urine, the larger stones are often removed by a procedure called a cystolitholapaxy. During the procedure, a cystoscope is inserted through urethra into the bladder. The endoscope carries an optical element for viewing stones and a working channel for the introduction of tools to the distal end of the device. Typically, a laser, ultrasound or mechanical device is used to break the stones into small pieces and flushes the pieces out of the bladder. Optionally, the intact stones and/or fragments may be removed by a deployable basket advanced through a working channel. Surgery has also been used to remove stones that are large or too hard to break up. Alternatively, the stones can be dissolved by chemical agents if the urinary pH can be made greater than or equal to 6.5. For example, urinary alkalization by potassium citrate is effective on the dissolution of uric acid stones. Renacidin can be used to dissolve phosphate or struvite calculi. Other rarely used agents include Suby G and M solution.

U.S. Pat. Pub. No.: 2010/0137846 to Desai et al. discloses a stone capture device comprises a shaft with a deployable sweeping structure at its distal end, as shown in FIG. 1. More particularly, the shaft is adapted to be removably placed over and connected to a conventional ureteroscope. The combination of the stone capture device and ureteroscope can be introduced into the urinary tract to capture, fragment, and remove stones from the bladder and kidney. Even though an apparatus is deployed to capture and remove bladder or kidney stones in Desai, it does not use an inflatable and adjustable component to capture stones and stones are not disrupted by chemical agents.

U.S. Pat. Pub. No.: 2013/0018314 to Teague et al. discloses medical procedures, devices and kits for the formation and removal of plug-forming compositions, as shown in FIG. 2. More particularly, the medical device comprises an elongated body, a proximal end and a distal tip. The device also comprises a lumen whose proximal end terminates at the proximal end and whose distal end terminates at the distal tip. The proximal end is adapted to engage a source of fluid. The lumen is adapted to carry carrying the fluid from the proximal end to the distal tip of the device. The distal tip is adapted to reverse the flow direction of fluid emerging from the catheter such that fluid emerging from the distal tip flows in the direction of the proximal end of the device.

U.S. Pat. Pub. No.: 2013/0178866 to McDougal et al. discloses a non-lithotripsic kidney-stone therapy, as shown in FIG. 3. More particularly, the method uses peristalsis to force a polymer plug through a mammalian lumen, thereby removing any calculi and/or calculi fragments present in the lumen.

U.S. Pat. No. 8,551,182 to Todd discloses devices, a system, and a method for removing kidney stones from the urinary system of a patient with reduced pain and expenses, as shown in FIG. 4. More particularly, the system includes expandable, elongated balloon sheaths that deliver and retrieve forceps and a urethral sparing prosthetic stent. Even though all abovementioned methods or devices are configured to capture, trap and removes stones, the stones cannot be effectively removed because they are not enclosed nor disrupted by chemical reagents before removal.

Therefore, there remains a need for a new and improved apparatus and method for effectively capturing and removing stones or stone fragments from urinary system without any future complication, especially for a large compartment such as the bladder.

### SUMMARY OF THE INVENTION

It is an object of the present invention to provide an apparatus and method to treat lithiasis, such as bladder stones, so that the process can be efficient, safe, and minimally invasive.

It is another object of the present invention to provide a medical apparatus and method to remove urinary stones in the bladder by trapping the stones in a confined space to dissolve or disrupt them.

It is a further object of the present invention to provide a medical apparatus assembly and method to remove urinary stones by introducing chemical reagents into the confined space created by the medical apparatus assembly to dissolve or disrupt the stones.

It is another further object of the present invention to provide a medical apparatus assembly and method to remove urinary stones from a body cavity, wherein the medical apparatus can be controlled by a computer.

In one aspect, a medical apparatus for lithiasis treatment may include an inflatable tubular component that can be inflated into a U-shaped structure, a plurality of flexible connecting portions formed on a main body. In one embodiment, the connecting portion serves as an intake port for inflating the inflatable tubular component, and another flexible tubular outlet serves as an intake port for inflating an inflatable cylindrical component formed in the U-shaped structure. The medical apparatus for lithiasis treatment may further include a plurality of control filaments disposed on an inner surface of the U-shaped structure to control the size thereof, so the inner surface of the U-shaped structure can tightly press against an outer surface of the inflatable cylindrical component when it is inflated, and a chamber is formed in the U-shaped structure next to the inflated cylindrical component. It is noted that the inflatable cylindrical component is integrated with the U-shaped structure. It is noted that the medical apparatus can be made by chemically synthetic materials such as PVC, natural rubbers, etc.

In another embodiment, the inflatable cylindrical component is separable from the U-shaped structure and it can be attached to the U-shaped structure when needed. One of the flexible connecting portions can be split into a inflating portion and an exhausting portion, wherein the inflating portion is used to inflate the inflatable cylindrical component, and the exhausting portion is configured to exhaust the fumes that may generated in the chamber after introducing chemical reagents therein.

When in use, the apparatus is introduced into a bladder, and once the bladder stones are found or located, the inflatable tubular component is inflated into the U-shape structure to trap the bladder stones by pumping in air or liquid through at least one of five flexible connecting portions. Meanwhile, the inflatable cylindrical component is also inflated into a cylindrical structure to further enclose the trapped stones in the chamber through at least one of five flexible connecting portions. After the stones are trapped in the chamber, one or more chemical reagents can be injected into the chamber through one of the three flexible connecting portions into the chamber to dissolve or disrupt the trapped bladder stones. Disrupted stones or fragments can be removed out of the bladder through one of the three flexible connecting portions.

In a further aspect, a computer can be used to control the medical apparatus. More specifically, the computer can be used to control the connecting portions to control the speed of inflation of the U-shaped structure and the cylindrical component. Also, the computer can control the flow rate and pH value of the chemical reagents introduced into the chamber. Also, the computer can be used to control the temperature of the apparatus and when an emergent situation occurs, the computer can be used to stop the stone removal process. In one embodiment, the computer is connected with a communication network to transmit out the parameters (inflation rate, pH value, pressure, temperature, etc.) during the stone removal process, so it can be monitored to ensure the safety of the process. The computer can also receive parameters from outside to perhaps enhance the stone removal process. In another embodiment, the computer can be connected with a mobile device, so the mobile device user, perhaps a medical profession can monitor the process or be more involved in the process if emergency occurs.

### BRIEF DESCRIPTION OF THE DRAWINGS

FIG. 1 illustrates a prior art disclosing a stone capture device comprises a shaft with a deployable sweeping structure at its distal end.

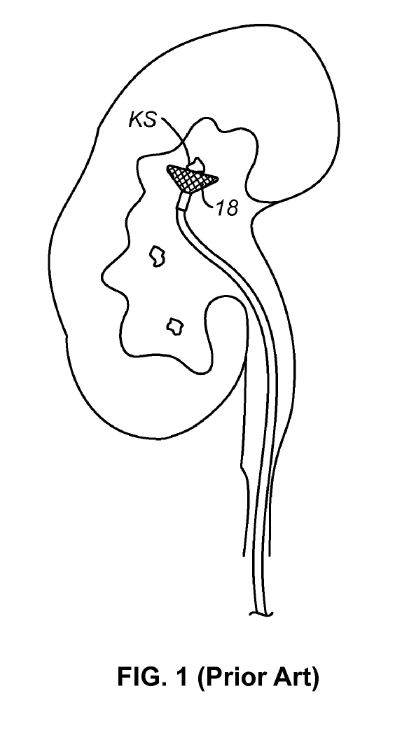

FIG. 2 illustrates a prior art disclosing medical procedures, devices and kits for the formation and removal of plug-forming compositions.

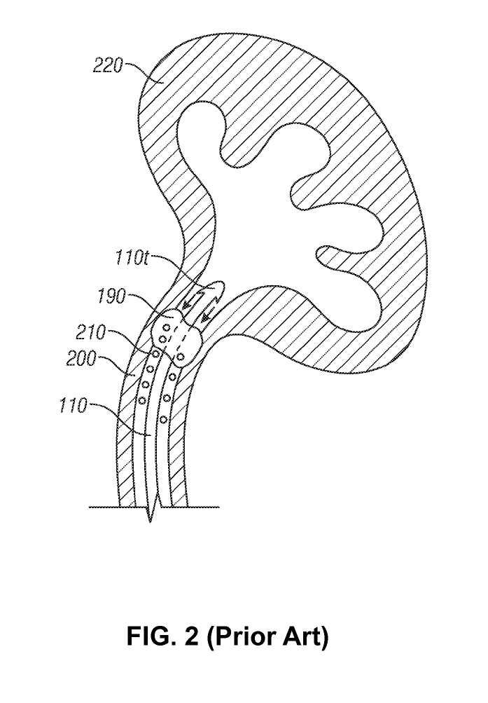

FIG. 3 illustrates a prior art disclosing a non-lithotripsic kidney-stone therapy.

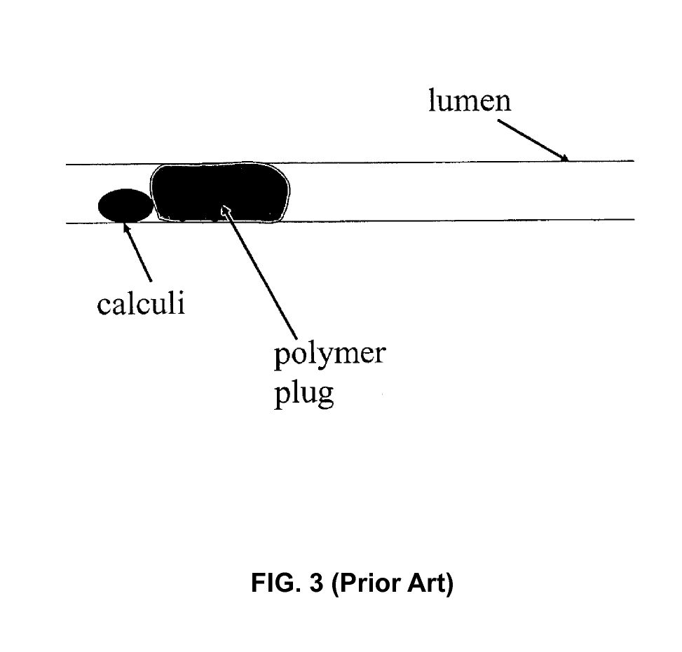

FIG. 4 illustrates a prior art disclosing devices, a system, and a method for removing kidney stones from the urinary system of a patient with reduced pain and expenses.

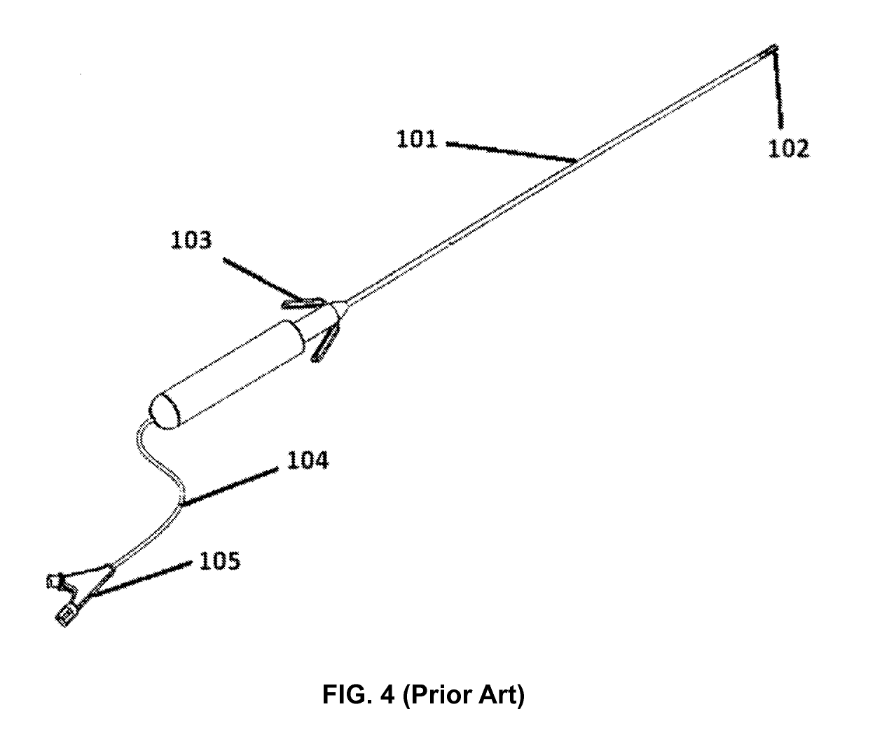

FIG. 5 illustrates one aspect of the medical apparatus for removing urinary stones from a body cavity in the present invention.

FIG. 6 illustrates the medical apparatus in the present invention when the U-shaped structure is formed to capture the stones.

FIG. 7 illustrates the medical apparatus in the present invention when the inflatable component is inflated to create a chamber to further process the stones.

FIG. 8 is an enlarged view of FIG. 7 illustrating the inflatable component and the chamber in the present invention.

FIG. 9 illustrates another embodiment disclosing a separable inflatable component in the present invention.

FIG. 10 illustrates another aspect of an auxiliary tube in the present invention.

FIG. 11 illustrates a further aspect in the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

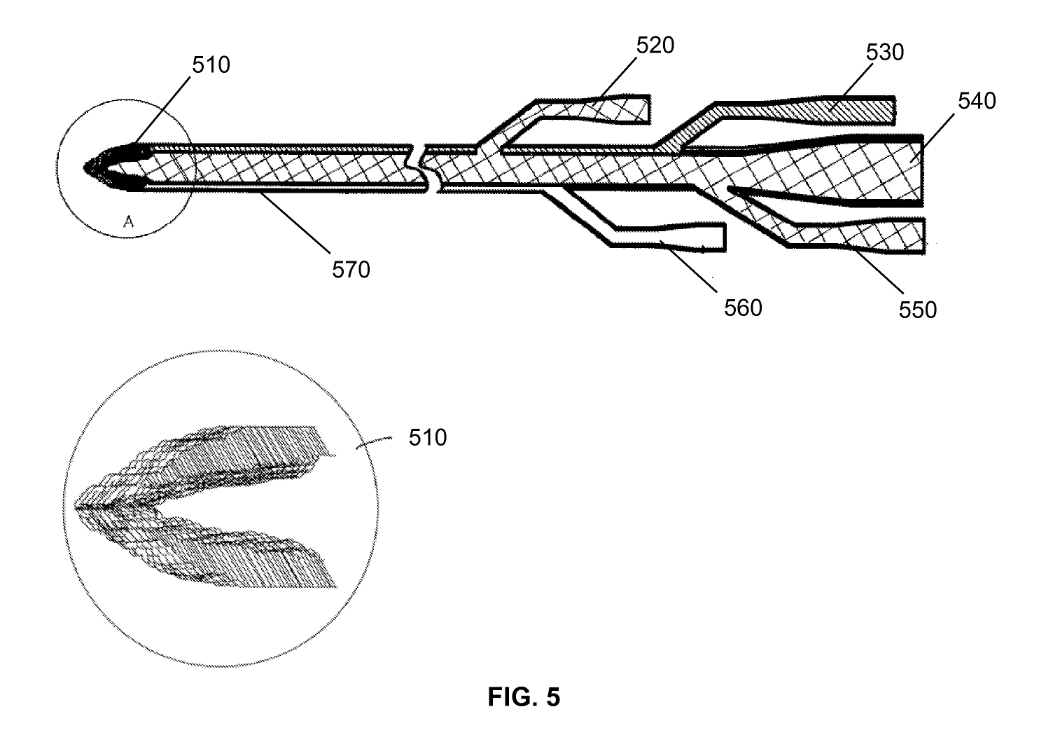

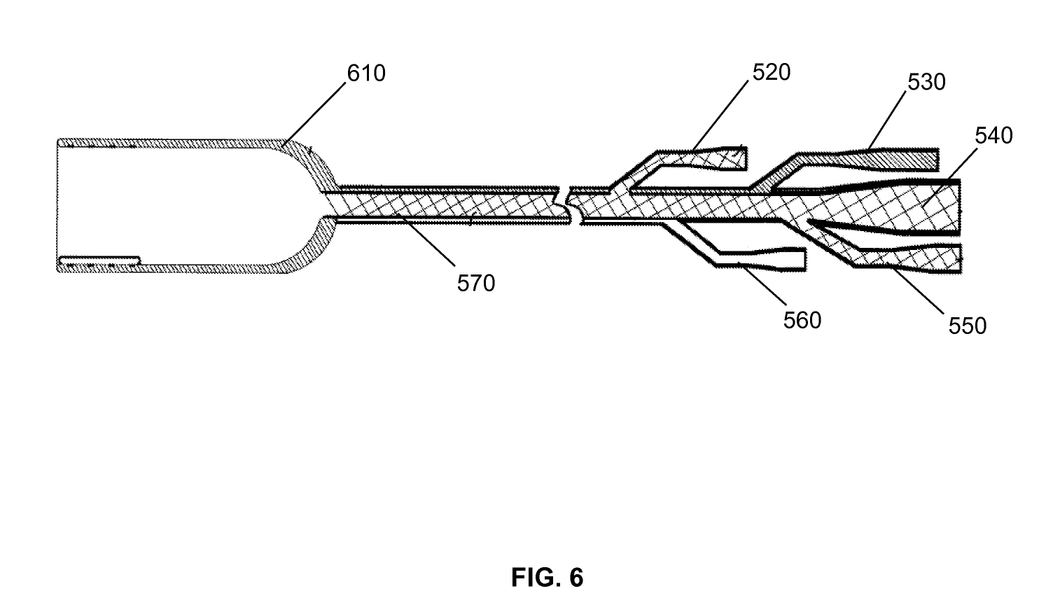

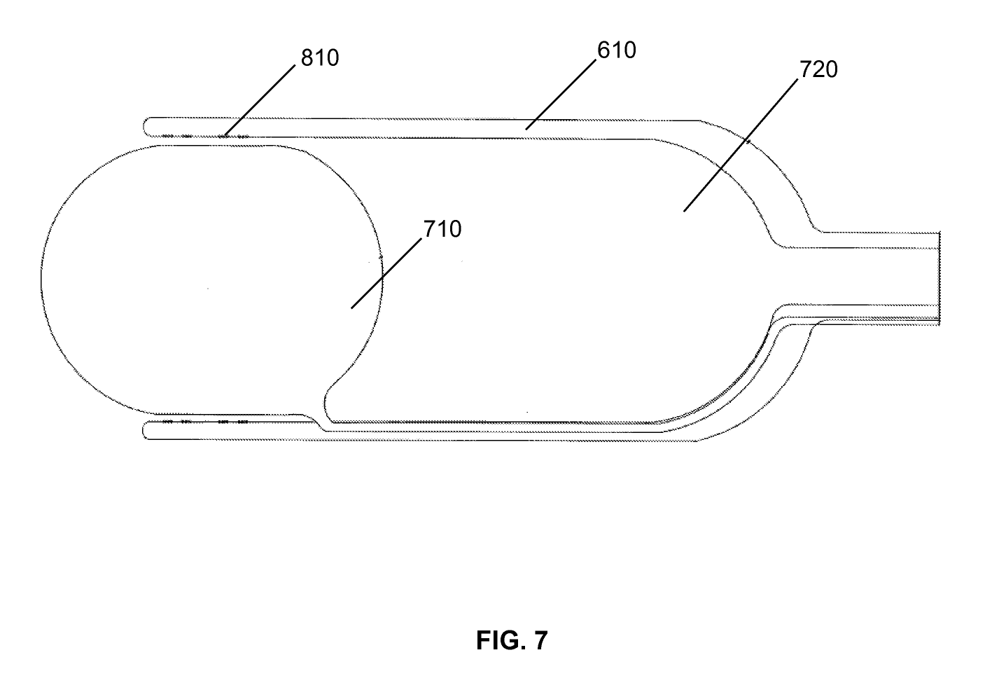

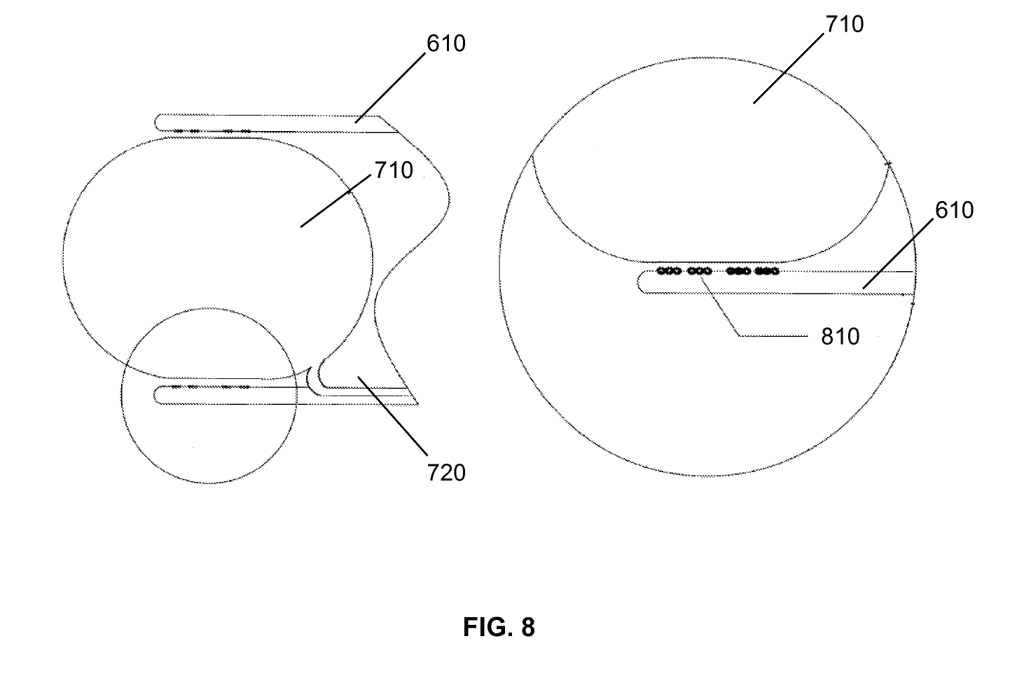

In one aspect, referring to FIGS. 5 to 8, a medical apparatus for lithiasis treatment 500 may include an inflatable tubular component 510 that can be inflated into a U-shaped structure 610, a plurality of flexible connecting portions 520, 530, 540, 550, 560 formed on a main body 570. In one embodiment, the connecting portion 530 serves as an intake port for inflating the inflatable tubular component 510, and another flexible tubular outlet 560 serves as an intake port for inflating an inflatable cylindrical component 710 formed in the U-shaped structure 610. The medical apparatus for lithiasis treatment 500 may further include a plurality of control filaments 810 disposed on an inner surface of the U-shaped structure 610 to control the size thereof, so the inner surface of the U-shaped structure 610 can tightly press against an outer surface of the inflatable cylindrical component 710 when it is inflated, and a chamber 720 is formed in the U-shaped structure 610 next to the inflated cylindrical component 710. It is noted that the inflatable cylindrical component 710 is integrated with the U-shaped structure 610. It is noted that the medical apparatus 500 can be made by chemically synthetic materials such as PVC, natural rubbers, etc.

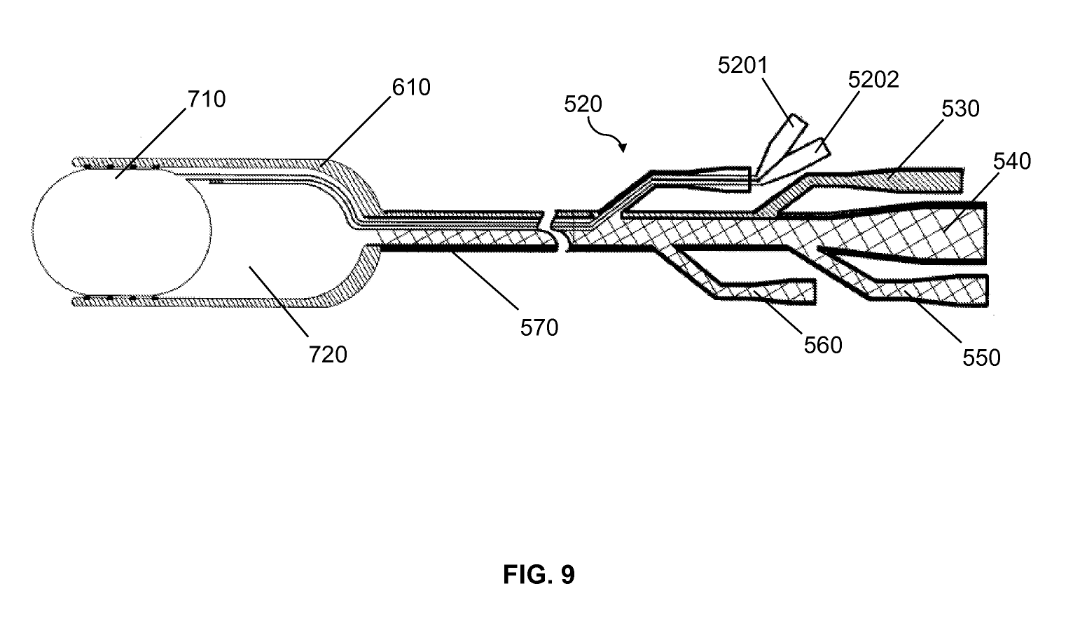

In another embodiment, as shown in FIG. 9, the inflatable cylindrical component 710 is separable from the U-shaped structure 610 and it can be attached to the U-shaped structure 610 when needed. One (e.g. 520) of the flexible connecting portions can be split into a inflating portion 5201 and an exhausting portion 5202, wherein the inflating portion 5201 is used to inflate the inflatable cylindrical component 710, and the exhausting portion 5202 is configured to exhaust the fumes that may generated in the chamber 720 after introducing chemical reagents therein.

When in use, the apparatus 500 is introduced into a bladder, and once the bladder stones are found or located, the inflatable tubular component 510 is inflated into the U-shape structure 610 to trap the bladder stones by pumping in air or liquid through at least one (e.g. 530) of five flexible connecting portions (520, 530, 540, 550, 560). Meanwhile, the inflatable cylindrical component 710 is also inflated into a cylindrical structure 710 to further enclose the trapped stones in the chamber 720 through at least one (e.g. 560) of five flexible connecting portions (520, 530, 540, 550, 560). After the stones are trapped in the chamber 720, one or more chemical reagents can be injected into the chamber 720 through one of the three flexible connecting portions (520, 540, 550) into the chamber 720 to dissolve or disrupt the trapped bladder stones. Disrupted stones or fragments can be removed out of the bladder through one of the three flexible connecting portions (520, 540, 550).

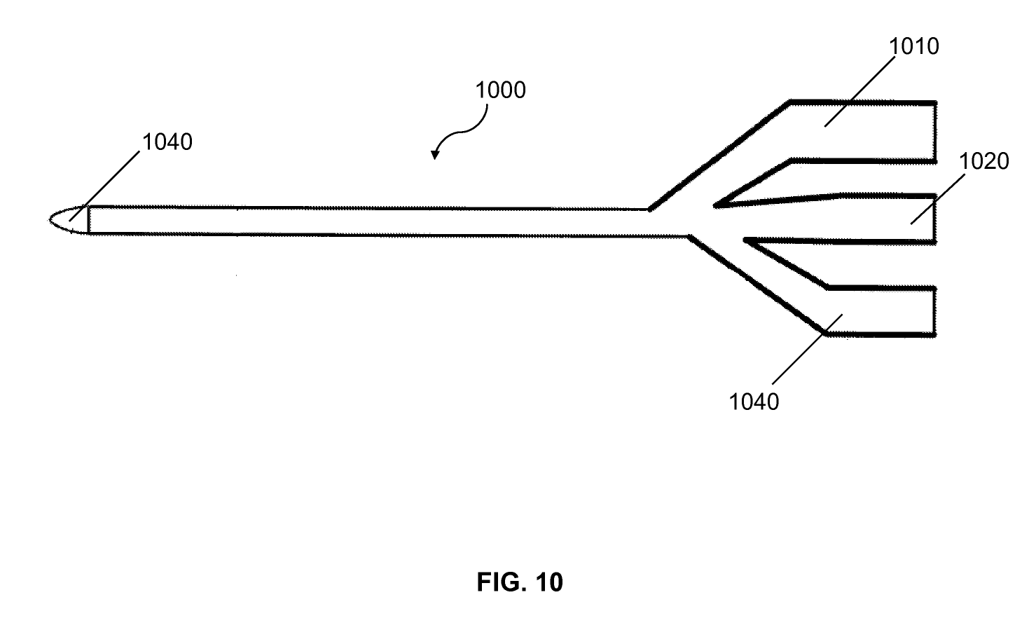

As mentioned above, since the medical apparatus 500 is made by chemical synthetic materials, namely “soft materials,” it may be difficult for the medical apparatus 500 to be introduced into the bladder. In another aspect, an auxiliary tube 1000 made by stainless steel can be used along with the apparatus 500 to resolve the problem. The auxiliary tube 1000 may include an opening 1010 for inserting the apparatus 500, an insertion tip control 1020, a urine outlet 1030 and an insertion tip 1040, as shown in FIG. 10. The insertion tip 1040 is located at the distal end of the auxiliary tube 1000 while the opening 1010, the insertion tip control 1020, and the urine outlet 1030 are at the proximal end thereof. When in use, the opening 1010 serves as a port for introducing the medical apparatus 500 into the bladder, and the insertion tip 1040 can be retracted by the insertion tip control 1020 after the apparatus is being inserted into the bladder. In one embodiment, the assembly of apparatus 500 and auxiliary tube 1000 are configured to capture bladder stones, confine them in the chamber 720, dissolve or disrupt the stones after chemical reagents are introduced, and finally remove the disrupted stones or stone fragments from the bladder.

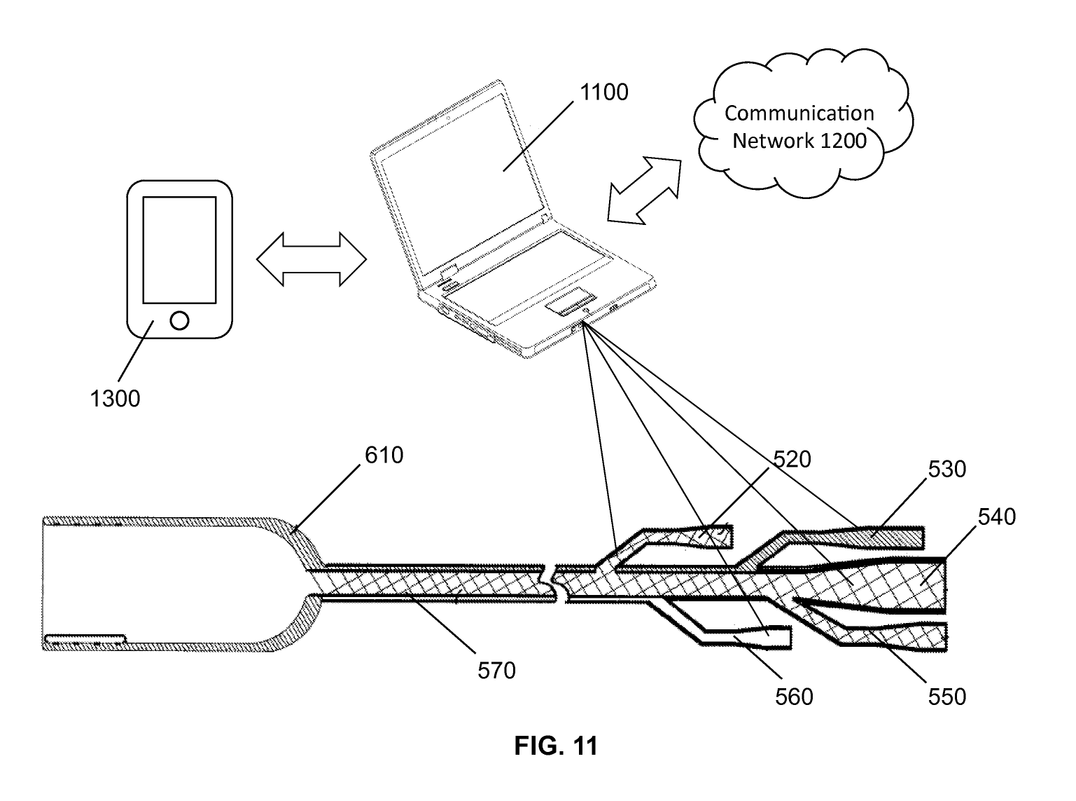

In a further aspect, a computer 1100 can be used to control the medical apparatus 500, as shown in FIG. 11. More specifically, the computer 1100 can be used to control the connecting portions 520, 530, 540, 550 and 560 to control the speed of inflation of the U-shaped structure 610 and the cylindrical component 710. Also, the computer 1110 can control the flow rate and pH value of the chemical reagents introduced into the chamber 720. Also, the computer can be used to control the temperature of the apparatus 500 and when an emergent situation occurs, the computer 1110 can be used to stop the stone removal process. In one embodiment, the computer is connected with a communication network 1200 to transmit out the parameters (inflation rate, pH value, pressure, temperature, etc.) during the stone removal process, so it can be monitored to ensure the safety of the process. The computer 1110 can also receive parameters from outside to perhaps enhance the stone removal process. In another embodiment, the computer 1100 can be connected with a mobile device 1300, so the mobile device user, perhaps a medical profession can monitor the process or be more involved in the process if emergency occurs.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. A medical apparatus comprising an inflatable tubular component that is configured to be inflated into a U-shaped structure, a plurality of connecting portions formed on a main body, wherein one of the connecting portions is used to inflate the inflatable tubular component, while the other connecting portion inflates an inflatable cylindrical component formed inside the U-shaped structure until an inner surface of the U-shaped structure tightly presses against an outer surface of the inflatable cylindrical component, and a chamber to trap bladder stones is formed in the U-shaped structure next to the inflated cylindrical component.

2. The medical apparatus of claim 1, further comprising a plurality of control filaments disposed on an inner surface of the U-shaped structure to control the size thereof.

3. The medical apparatus of claim 1, wherein the inflatable cylindrical component is detachable from the U-shaped structure.

4. The medical apparatus of claim 2, wherein the inflatable cylindrical component is detachable from the U-shaped structure.

5. The medical apparatus of claim 1, wherein the medical apparatus is made by chemically synthetic materials.

6. The medical apparatus of claim 3, wherein the connecting portion includes an inflating portion used to inflate the inflatable cylindrical component, and an exhausting portion configured to exhaust the fumes generated in the chamber after introducing chemical reagents therein.

7. The medical apparatus of claim 1, wherein a computer is used to control the medical apparatus by connecting said computer to the connecting portions.
